(19) United States
(12) Patent Application Publication
(10) Pub. No.: US 2025/0130322 A1
(43) Pub. Date: Apr. 24, 2025

(54) COMPACT MIXED ARRAY IMAGING PROBE

(71) Applicant: DEEPSIGHT TECHNOLOGY, INC., Santa Clara, CA (US)

(72) Inventors: Yihang LI, St. Louis, MO (US); Linhua XU, University City, MO (US); Lan YANG, Clayton, MO (US); Michael HAZARIAN, San Jose, CA (US); Haochen KANG, Sunnyvale, CA (US)

(21) Appl. No.: 18/923,071

(22) Filed: Oct. 22, 2024

Related U.S. Application Data
(60) Provisional application No. 63/545,327, filed on Oct. 23, 2023.

(51) Int. Cl. G01S 7/52; A61B 8/00; A61B 8/12

(52) U.S. Cl. CPC G01S 7/52023; A61B 8/12; A61B 8/4488; G01S 7/52057

(57) ABSTRACT

A compact mixed ultrasound transducer including a transducer case, a source array configured comprising at least one acoustic energy generating transducer and configured to transmit acoustic waves; and an optical receiver array including at least one optical sensor and configured to detect acoustic echoes associated with the acoustic waves is provided. The mixed ultrasound transducer may have a compact geometry facilitated by various features.

### RELATED APPLICATIONS

This application claims priority to U.S. Provisional Application No. 63/545,327, titled Miniature Mixed Array Imaging Probe, filed Oct. 23, 2024, which is incorporated herein by reference.

### TECHNICAL FIELD

The invention relates generally to the field of ultrasound sensing and imaging.

### BACKGROUND

The present disclosure relates to, without limitation, optical sensing, particularly to a design and packaging of an optical-acoustic miniature mixed array imaging probe. In certain medical diagnostic and therapeutic procedures, medical personnel will utilize intraoperative ultrasound imaging to image the area of the procedure. Ultrasound imaging is known to be an advantageously non-invasive form of imaging, as it is not based on ionizing radiation. For example, a doctor may use endoscopic, intravascular or endoluminal ultrasound to view the inside of a person's body.

During an endoscopy or endoluminal procedure, an endoscope or scope can be inserted into a person's body through the mouth, gastrointestinal tract or other body lumen or through an incision in order to access a body cavity, such as joints, brain or abdomen or a lumen, such as with intravascular ultrasound. Conventional scopes are tubes that may have a light and/or camera affixed to an end (e.g., a distal end) that is first inserted in the body, which allows the doctor to see internally.

Some scopes include an ultrasound transducer mounted to its distal end in addition to or instead of the light and/or the camera. Such devices are used to perform endoluminal ultrasound.

Such devices use sound waves to produce images of the body, such as the digestive track and surrounding organs and tissues, including the lungs, pancreas, gall bladder, liver, vasculature, cranial and lymph nodes. Some existing imaging technologies use Acoustic Energy Generating (AEG) materials for transducers to generate imagery during a diagnostic or therapeutic medical procedure. Commonly used AEG materials include piezoelectric materials such as lead-zirconate-titanate (PZT), ceramic, piezoelectric single crystal (e.g., PIN-PT, PIN-PMN-PT), and polyvinylidene fluoride (PVDF) among other materials known to those of skill in the art. AEG transducers have limitations, especially when a small form factor is needed as small AEG transducers generally have low to minimal signal output. Therefore, it may be challenging to use AEG transducers for medical applications constrained to a small form factor because of the size limitations (e.g., physical size limitations).

### SUMMARY

Various examples and embodiments are described in relation to a miniature mixed array imaging probe. These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

Systems, devices, and methods for ultrasound sensing and imaging are presented herein. In particular, systems, devices, and methods described herein may include imaging transducers that include acoustic energy generating transducers and optical fiber based ultrasound receiving devices. Further, these mixed ultrasound transducers are configured to have a compact geometry, as discussed further herein.

In embodiments, a compact mixed ultrasound transducer is provided. The compact mixed ultrasound transducer includes a transducer case, a source array configured comprising at least one acoustic energy generating transducer and configured to transmit acoustic waves, and an optical receiver array including at least one optical sensor and configured to detect acoustic echoes associated with the acoustic waves.

### BRIEF DESCRIPTION OF DRAWINGS

The accompanying drawings, which are incorporated into and constitute a part of this specification, illustrate one or more certain examples and, together with the description of the example, serve to explain the principles and implementations of the certain examples.

FIGS. 1A-1C illustrate embodiments for various configurations of an optical fiber sensor for use in a compact mixed ultrasound transducer.

FIG. 1D illustrates geometry aspects of a compact mixed ultrasound transducer.

FIGS. 2A and 2B illustrate embodiments for various configurations of an optical fiber sensor for use in a compact mixed ultrasound transducer.

FIG. 2C illustrates an example of an optical receiver array disposed on a substrate.

FIGS. 3A-3D illustrate an embodiment of a compact mixed ultrasound transducer.

FIG. 4 is an illustration of an embodiment of a compact mixed ultrasound transducer.

FIGS. 5A-5D illustrates an embodiment for a design of a side-view compact mixed ultrasound transducer.

FIGS. 6A-6C illustrate design variations for optical I/O arrangements.

FIG. 7 illustrates an embodiment of a design for a side-view compact mixed ultrasound transducer.

FIGS. 8A-8D illustrate an embodiment of a design for a radial-view compact mixed ultrasound transducer.

FIGS. 9A-9B illustrate an embodiment of a multi-core fiber as a variation for optical input/output.

FIGS. 10A and 10B illustrate an embodiment of a compact mixed ultrasound transducer including collocated transducers.

FIGS. 11A and 11B illustrate an embodiment of a compact mixed ultrasound transducer including collocated transducers.

FIG. 12 illustrates a cable strain relief configuration.

FIG. 13 illustrates an embodiment of a circumscribed hexagon of a fiber, which represents an area taken by the fiber.

FIG. 14 illustrates an example ultrasound system compatible with embodiments hereof.

FIG. 15 illustrates an embodiment of a method using a compact mixed ultrasound transducer.

Similar reference characters denote corresponding features consistently throughout the attached drawings.

### DETAILED DESCRIPTION

Examples are described herein in the context of a miniature mixed array imaging probe. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of an actual implementation, numerous implementation-specific decisions will be made in order to achieve the developer's specific goals, such as compliance with medical application- and business-related constraints, and that these specific goals will vary from one implementation to another and from one developer to another.

Conventional ultrasound sensing uses Acoustic Energy Generating (AEG) materials for transducers. Commonly used AEG materials include piezoelectric materials such as lead-zirconate-titanate, ceramic, piezoelectric single crystal (e.g., PIN-PT, PIN-PMN-PT), and polyvinylidene fluoride (PVDF) among many other materials known to those of skill in the art. However, some of the challenges associated with use of piezoelectric properties of these materials include a high operational voltage requirement, a high electric field requirement (which may cause breakdown and failure), a non-linear response with high hysteresis, and a limited angle of detection. Thus, there is a need for new and improved devices and methods for ultrasound sensing. Further, such devices are typically relatively large in size, as discussed in greater detail below. The present disclosure generally relates to the field of ultrasound, and particularly to methods and devices that enable ultrasound transducing using a mixed array including, such as integrating an array of optical sensors and other transducers.

Various known ultrasound transducers used in ultrasound imaging have numerous drawbacks. For example, some ultrasound transducers are made of piezoelectric material, such as PZT. However, the 6 dB bandwidth of PZT materials is generally limited to only about 70%.

Certain composite PZT materials may have a slightly increased bandwidth, but still only achieve a bandwidth of up to about 80%. As another example, single-crystal materials have increasingly been used in an effort to improve performance of ultrasound probes, but single-crystal materials have lower Curie temperatures and may be brittle. Another type of transducer material is silicon, which can be processed to build Capacitive Micromachined Ultrasound Transducer (CMUT) probes that can have increased bandwidth. However, CMUT probes are not very sensitive or reliable. Moreover, CMUT probes have several operational limitations. For example, CMUT probes are nonlinear transducers and, therefore, are not generally suitable for harmonic imaging. Additional, CMUT probes use an additional bias voltage to operate properly. Thus, there is a need for improved devices and methods for ultrasound transducing.

In some configurations, an apparatus for imaging a target may include an ultrasound transducer array that includes one or more array elements of a first type and one or more array elements of a second type different from the first type. The first type may be a transducer (e.g., AEG materials including, for example, piezoelectric transducers or capacitive micromachined ultrasonic transducers (CMUT)) configured to transmit acoustic waves, and the second type may be an optical sensor (e.g., an interference-based optical sensor such as an optical resonator, an optical interferometer, etc.). The array elements of the first and second types are configured to detect acoustic signals (e.g., echoes) scattered, reflected or generated within the field of view (FoV) from the transmitted acoustic waves.

In some configurations, a mixed ultrasound transducer includes an AEG material subarray and an optical receiver array (ORA, or optical subarray for short). An optical subarray may be realized in various platforms, e.g. photonic integrated circuits (PIC), fiber-based platform, etc. ORAs consistent with embodiments herein may have one or more acoustic optical sensors, e.g., 1 or more, 2 or more, 3 or more, 4 or more, 5 or more, 10 or more, 20 or more, 100 or more, etc. In some embodiments, an ORA consistent with embodiments hereof includes 30-48 optical elements.

In some configurations, a system comprising a mixed imaging transducer contains an ultrasound AEG material element/subarray and an optical receiver element/subarray (optical sensor/receiver/subarray for short). The AEG/ORA arrays and subarrays are compact so that they can be mounted in a catheter and coupled at or near the end of an endoscope/endoluminal. The transducer can thus be used in conjunction with other endoscopic/endoluminal surgical, IVUS (intravascular ultrasound) systems or diagnostic instruments

A compact mixed ultrasound transducer with a very compact footprint, with a cm-level or sub-cm imaging aperture (miniaturized mixed transducer) may be specifically designed for use on the distal end of a medical device used in vivo such as for intravascular ultrasound, endoscopic/endoluminal ultrasound, laparoscopic surgery ultrasound, robotic surgical tools & arms and other intraoperative tools or hand-held tools used for ultrasound imaging and requiring similar small footprint. The compact mixed ultrasound transducer may include multiple sub-arrays, including at least one or more AEG transducer array and one or more optical receiver array. A transducer in the AEG transducer array may be, for example, a piezoelectric transducer or capacitive micromachined ultrasonic transducer (CMUT)) configured to generate and/or transmit acoustic waves. The compact mixed ultrasound transducer may also include an optical sensor, such as an optical receiver array (ORA), which may be mounted at least partially in the compact mixed ultrasound transducer case or housing, which may be mounted in a catheter. In some configurations, a photodiode that converts optical signals into electrical signals of the sensor may be located outside of the compact mixed ultrasound transducer case or housing, e.g., at a proximal end of a catheter (or other medical device) in which the compact mixed ultrasound transducer is accommodated. Such a photodiode may be disposed anywhere within or outside of the medical device including the compact mixed ultrasound transducer that provides a large enough area to accommodate the electronics, thermal management and EM shielding that are required for low-noise photodetection and transmission of the electrical signal. The ORA array may comprise, for example, one or multiple optical ultrasound sensors such as interference-based optical sensors, such as optical resonators, Fabry-Perot cavities, optical interferometers, etc., and/or other sensors based on other signal transduction mechanisms. The ORA may be fabricated from a chip/wafer-based platform or a fiber (array) platform. Examples of such arrays may be found, for example, as described in U.S. patent application Ser. No. 18/609,378, titled “Fiber-Optical Sensor Array for Sensing and Imaging” and filed on Mar. 19, 2024 and U.S. application Ser. No. 18/597,493, filed Mar. 6, 2023 titled “MIXED ARRAY PROBE.” The ORA may be configured to detect acoustic returned or reflected signals corresponding to transmitted acoustic waves. The acoustic returned/reflected signals are in turn used to generate an ultrasound image corresponding to the anatomy of the subject.

Although the following description references a single compact mixed ultrasound transducer disposed on a medical device, the invention is not limited to such. In embodiments, one, two, or more compact mixed ultrasound transducer may be included within a single medical device (e.g., an endoscope). In embodiments, two or more compact mixed ultrasound transducers may be of different types as described herein.

The compact mixed ultrasound transducer is configured to propagate acoustic waves along a particular plane relative to the compact mixed ultrasound transducer. This plane corresponds to an image slice, which may be of varying thickness. The acoustic waves may propagate along the plane while the image slice associated with the plane may extend above and below the plane, depending, for example, on the properties of the ultrasound transducer elements used to generate the acoustic waves. For instance, the compact mixed ultrasound transducer may be shaped as a cylinder with a distal end and a length. In some configurations, a compact mixed ultrasound transducer may propagate acoustic waves and thereby produce an image slice that extends from the tip of the distal end of the compact mixed ultrasound transducer (front view). In embodiments, the image slice may have a thickness or height depending on the ultrasound transducers and may widen with respect to the ultrasound transducers as it extends away from the distal end. In some configurations, the compact mixed ultrasound transducer may detect an image slice that extends from the length of and is parallel to the compact mixed ultrasound transducer (side view). In embodiments, the image slice may have a thickness or height depending on the ultrasound transducers. The image slice in such an embodiment may correspond to side face of the transducer and/or may expand or widen at distances away from the side face. In some configurations, the compact mixed ultrasound transducer may be configured to detect an image slice that is perpendicular to and extends radially around the length of the compact mixed ultrasound transducer (radial view). In embodiments, the image slice may have a thickness or height depending on the ultrasound transducers. Such a slice may expand as it extends away from the compact mixed ultrasound transducer. The compact mixed ultrasound transducer may include one or more acoustic interfaces. The acoustic interface may provide mechanical protection of the acoustic transducer array and/or the optical receiver array. The acoustic interface may also manage the acoustic field of the source and/or the echo for better imaging reconstruction. The acoustic interface may create an acoustic channel between the transducer and optical receiver sub-arrays to help ensure acoustic conduction between the arrays and a region of interest in an imaging field.

Embodiments herein provide compact mixed ultrasound transducers. Such transducers present many engineering and fabrication challenges. In embodiments, mixed ultrasound transducers are configured to be operational with catheters, and may therefore have a diameter in the range of approximately 1 to 8 mm, 1 to 6 mm, and/or 2 to 5 mm. Diameters that are too large can create challenges when trying to navigate such catheters through small body lumens. Further, transducers consistent with embodiments hereof may be compact in an axial direction as well, as longer transducers (which may have rigid cases) may be more difficult to navigate through body lumens. In examples, transducers consistent with embodiments hereof may have an axial length of between 1 and 6 mm, between 2 and 5 mm, and/or between 2 and 4 mm. Creating such a compact transducer for use in a catheter based application requires solving several problems, as discussed below.

First, the transducer arrays themselves must be compact. Solutions to this challenge may be facilitated through the use of optical sensor arrays, as discussed herein, due to their very small size. Further, such optical sensor arrays may be collocated with AEG source arrays, allowing for an even more compact overall footprint.

Second, input and output connections to the transducer arrays must be managed and kept small to ensure that the cross-section of the catheter on which the mixed ultrasound transducer is mounted is not too large. Solutions to this challenge may be facilitated through the use of one of more multi-core fibers to reduce the total number of optical fibers acting as input/output to the sensors of the optical array. Further, wavelength division multiplexing may be used so that a single optical waveguide can carry the input/output optical signal for multiple optical sensors.

Next, electrical and optical traces within the transducer may be kept to a minimum size, a minimum number, and/or may be routed in a manner that reduces size to ensure that the transducer case remains within the appropriate size tolerances.

Further, the layout of the AEG source arrays and optical receiving arrays may be optimized to for the purposes of space efficiency to maintain a compact transducer case.

Each of the above, and additional features discussed herein, represent technical solutions to technical problems that arise when designing and fabricating novel mixed ultrasound transducer arrays as discussed herein.

The following illustrative example is given to introduce the reader to the general subject matter discussed herein and the disclosure is not limited to this example. The following sections describe various additional non-limiting examples and examples of a miniaturized mixed array ultrasound probe.

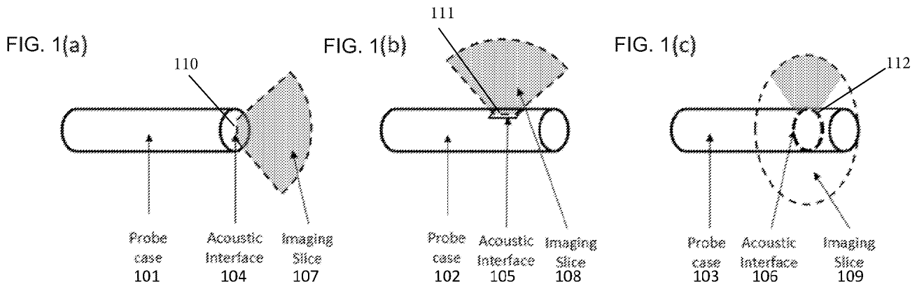

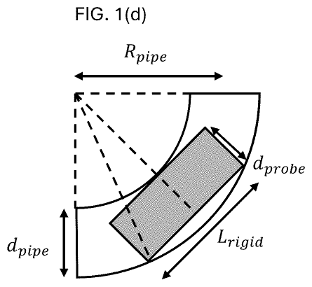

Turning to the Figures, FIGS. 1A-1C are schematic drawings illustrating three example configurations of a miniaturized compact mixed ultrasound transducer consistent with embodiments hereof: (a) front-view; (b) side-view; (c) radial view. The compact mixed ultrasound transducers shown in FIGS. 1A-1C are configured to be coupled to an endoscope and/or any other suitable medical device or surgical tool. For the purposes of explanation, an endoscopic application will be discussed, but it is understood that these probes are not so limited and all references to an endoscopic catheter or the like may be assumed to also include any other suitable medical device. The three configurations (a, b, c) of the miniaturized probe support different imaging scenarios: front-view (a), side-view (b), and radial-view (c). In some endoscopic applications, the compact mixed ultrasound transducer and the cables may be encapsulated in a cylindrical-shape transducer housing or case 101, 102, and 103. The size of the cross-section of the probe case 101, 102, and 103 is determined by the application, e.g., the particular anatomy, organ, lumen, cavity etc., in which the endoscopy is to be performed.

In the examples shown, an acoustic wave or signal is generated by an AEG transducer array and then transmitted through the acoustic interface 104, 105, and 106. The acoustic interfaces illustrated in FIGS. 1A-1C depict an example of a minimum imaging window 110, 111, 112 and the relative position of the window to the probe case. In various examples, the acoustic interface may incorporate additional structures outside the compact mixed ultrasound transducer case or housing case. The shape, location, and orientation of the acoustic interface determines the shape and direction of the imaging slice 107, 108, and 109.

In various examples, different types of probes may use different designs of the compact mixed ultrasound transducer. Various aspects of these designs, including, for example, the following aspects, are described below: (1) a optical sensor or sensor array; (2) subarray integration and assembly; (3) acoustic interface and source array; and (4) cable management for different cases.

Compared to conventional ultrasound imaging probes, mixed ultrasound imaging probes can provide better imaging resolution, deeper penetration, and/or support more imaging modes (e.g., multiple harmonic imaging modes) operating simultaneously. Providing such improvements to a mixed-array probe configured to be used in conjunction with endoscopic procedures is particularly advantageous. For example, as an AEG transducer array is reduced in size (e.g., for a smaller form factor), a strength of transmitted acoustic waves is reduced. By using an optical receiver array, sensitivity is increased for detecting acoustic signals corresponding to weaker transmitted acoustic waves.

In some configurations, the ORA may be a chip-based, photonic integrated circuit (PIC) optical receiver array including one or more optical sensors. Examples of such a chip are described in, for example, U.S. application Ser. No. 18/429,517, filed Feb. 1, 2024 titled OPTICAL SENSOR CIRCUIT AND OPTICAL SENSING METHOD and U.S. application Ser. No. 18/597,493, filed Mar. 6, 2023 titled MIXED ARRAY PROBE, the disclosures of which are incorporated by reference for all purposes.

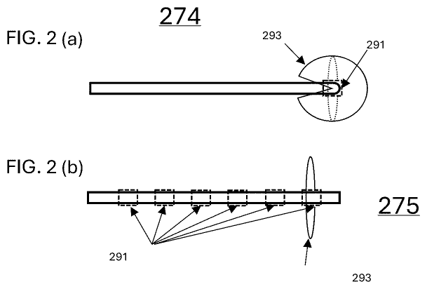

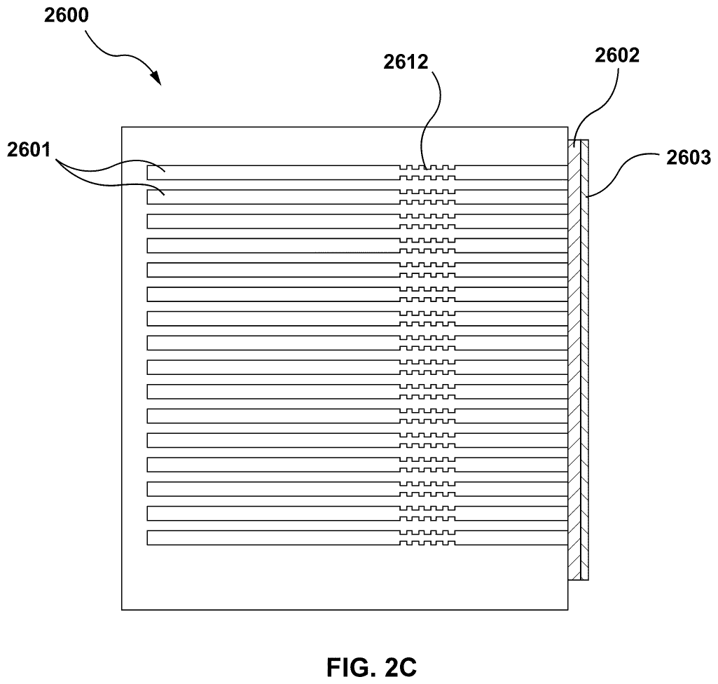

FIG. 2C illustrates a chip based optical receiver array consistent with embodiments hereof. FIG. 2C illustrates an on-chip fiber optical sensor array 2600 that may include an array of fiber optical sensors formed together on a single chip. In an embodiment, the on-chip fiber optical sensor array 2600 may include a plurality of optical waveguides 2601 (which may be fiber optic cores, for example) sharing a distal reflecting surface 2603 and an acoustically sensitive polymer portion 2602. The distal reflecting surface 2603 and an acoustically sensitive polymer portion 2602 stretch across the entirety of the sensor array 2600. The distal reflecting surface 2603 and the sensitive polymer portion 2602 may react mechanically or have their properties changed by incident acoustic waves, thereby causing optical signals reflected therefrom to change in a fashion that may be interpreted for ultrasound imagery, as described herein. In further embodiments, any suitable fiber end optical sensor structure of the current disclosure may be suitable for the on-chip fiber optical sensor array 2600. In embodiments, each of the optical waveguides 2601 may include a Bragg grating 2612. In embodiments, the fiber optical sensor array 2600 may be manufactured by mounting individual optical fibers to the substrate, optionally performing one or more operations to smooth the faces of the optical fibers, and then applying the distal reflecting surface 2603 and the acoustically sensitive polymer portion 2602. In embodiments, the fiber optical sensor array 2600 may be manufactured, e.g., through the use of UV lithography or other suitable additive manufacturing technique to write the optical waveguides 2601 to create PICT. Accordingly, in embodiments, at least a portion of the fiber optical sensor array 2600 may be formed of the chip (e.g., the substrate) itself. In embodiments, the distal reflecting surface 2603 and the acoustically sensitive polymer portion 2602 may be applied continuously across the multiple optical waveguides. In embodiments, each optical waveguide 2601 may have a distal reflecting surface 2603 and acoustically sensitive polymer portion 2602 applied individually. Each optical waveguide 2601 may function as an individual optical sensor.

The orientation of on-chip fiber optical sensor array 2600 may be such that acoustic waves are incident on distal reflecting surface 2603. Alternatively, the on-chip fiber optical sensor array 2600 can be oriented such that the acoustic waves are incident along the length of the distal portion of the optical waveguides 2601, including the Bragg gratings 2612. In such an arrangement, the Bragg gratings may be acoustic sensitive Bragg gratings such as disclosed in pending U.S. application Ser. No. 18/749,712, titled Optical Fiber with Acoustically Sensitive Fiber Bragg Gratings, filed Jun. 21, 2024.

Although many of the embodiments herein are discussed with respect to PIC optical receiver arrays, ORAs consistent with embodiments hereof may also be fabricated from a plurality of individual fiber sensors. In some configurations, the PIC optical receiver array includes one or more optical sensors used to detect acoustic waves. For example, the PIC optical receiver array may include one or more optical path (e.g., optical waveguides) with one or more optical features, such as phase velocity, polarization, etc., that change in response to an incident acoustic wave. The PIC optical receiver array can be used to detect the acoustic wave by transmitting, from an optical light source, optical light through the optical path and detecting, by an optical light receiver, changes in the light reflected by the optical features in response to changes in the one or more optical features. For example, a change in pressure on the optical waveguide induced by the acoustic wave can cause a change in refractive index of the optical waveguide by deforming the waveguide. When light transmitted through the PIC optical receiver array is reflected and received at an optical light receiver, these changes may be detected and used to determine ultrasound information (e.g., for generating an ultrasound image).

In embodiments, individual optical sensors of the ORA (e.g., individual fiber sensors or individual sensors of a PIC, each optical waveguide 2601) may be as small as approximately 10 μm, in embodiments, individual optical sensors may have a diameter between 50-250 μm or between 70 to 140 μm.

In some configurations, a chip-based optical receiver array (or individual fiber based ORA) may be less than a millimeter thick, less than 0.9 mm thick (e.g., in an elevation dimension), less than 0.8 mm thick, less than 0.7 mm thick, or approximately 0.6 mm thick. In embodiments, an ORA (PIC based or individual fiber based) may include 30-48 elements at a pitch of 100 μm, resulting in a lateral dimension of approximately 3 mm to 4.8 mm. In some embodiments, an ORA may include 8 elements and therefore have a lateral dimension less than 1 mm, e.g., approximately 0.8 mm. The substrate of a chip for the optical receiver may be single-crystal silicon, glass, sapphire, or another suitable material. The acoustic impedance of a substrate that significantly mismatches that of human body (water, fat, muscle, organs, etc, whose acoustic impedance lies within ±10% of water's) can cause reflection of acoustic power by more than 50% (e.g., 64% for tissue to glass, 74% for tissue to silicon, 88% for tissue to sapphire, etc.). Accordingly, in some configurations, the optical receiver may include a matching layer and an absorbing backing block, which may increase the overall thickness of the optical receiver. The matching layer provides an acoustic coupling layer to increase coupling efficiency from the tissue to the optical sensing layer by suppressing an acoustic reflection. For example, the matching layer may have an acoustic impedance between that of tissue and the substrate of the optical receiver to provide a transition material that reduces the impedance mismatch and therefore reduces the reflected power. The matching layer can be designed for increased efficiency for an acoustic frequency band of interest through materials selection, thickness adjustments, and multi-layering. In some configurations, a matching layer is not used (e.g., if an extremely broad acoustic bandwidth is designed for). The backing block may provide a mechanical support to the optical receiver array and may be designed to be acoustically matched to the receiving array substrate and acoustically absorptive, so that reduced or minimum acoustic reflection occurs from a backside of the receiving array. For front-view probes, such as the example shown in FIG. 1(a), the thickness of the optical receiver may depend on a steerability constraint of the endoscopic catheter (or other medical device in which the optical receiver array is deployed), because a thicker optical receiver, e.g., caused by stack-up of components, may not be as flexible as a thinner optical receiver. Referencing FIG. 1D, the rigid length of the compact mixed ultrasound transducer (e.g. corresponding to the thickness of the optical receiver array) may be mathematically constrained as follows:

$$0.5 {L}_{r i g i d} < \sqrt{{( {R}_{p i p e} + 0.5 {d}_{p i p e} )}^{2} - {( {R}_{p i p e} - 0.5 {d}_{p i p e} + 0.5 {d}_{p r o b e} )}^{2}}$$

In the above formula, R_{Pipe} represents a bending radius of a tube or pipe within which an endoscopic catheter (or other medical device) is constrained. dₚᵢₚₑ represents a diameter of a tube or pipe within which the endoscopic catheter is deployed. d_{probe} represents a diameter of the endoscopic catheter itself while L_{rigid} represents the rigid length of the compact mixed ultrasound transducer. The above formula may be understood as a guideline, as various geometries of bends in the deployment tube/pipe may require different constraints. In embodiments, as discussed above, L_{rigid} may be between 1 and 6 mm, between 2 and 5 mm, and/or between 2 and 4 mm.

Although ORAs as discussed herein may be fabricated in a more compact manner than AEG transducer arrays as used herein, AEG source transducer arrays consistent with embodiments hereof may also have a compact footprint. In embodiments, AEG transducers may be sized according an acoustic pitch required for a selected center frequency. In an embodiment, a pitch of approximately 100 um may be selected to achieve a center frequency at approximately 6-8 MHz. An AEG transducer source array consistent with embodiments hereof may include 30-48 elements and may have a lateral size of approximately 3-4.8 mm and an elevation size of approximately 1.1-1.2 mm.

For a side-view (FIG. 1(b)) and radial-view (FIG. 1(c)) compact mixed ultrasound transducer, the thickness of the optical receiver array may be constrained by the size (diameter) of the endoscopic catheter or other medical device within which the compact mixed ultrasound transducer is disposed. Such constraints are discussed further herein.

In some examples of the compact mixed ultrasound transducer, both the transducer (source) array and the receiving array may be one-dimensional. Typically, in such examples, the image quality may be improved by providing a large imaging aperture in the lateral direction.

Therefore, in an example using a PIC, the sensors of the ORA may substantially occupy the lateral width of the substrate chip, while the rest of the chip area may be assigned to routing of optoelectrical signals (e.g., traces) and the inputs and outputs (I/Os) (e.g., ports or connections). Various layouts of different types of compact mixed ultrasound transducers are described below. Though some examples may show a one-dimensional array, two-dimensional arrays may also be used in some configurations.

In some configurations, the optical sensors are fibers based. Examples of such a fiber-based sensor may be found in U.S. Pat. No. 12,025,489 issued Jul. 2, 2024 titled Fiber-Optical Sensor System for Ultrasound Sensing and Imaging and U.S. patent application Ser. No. 18/609,378 filed Mar. 19, 2024 and titled Mixed Transducer Array with Fiber Sensors, the disclosures of which are incorporated by reference for all purposes. Fiber-based sensors include optical waveguides in their core and are configured such that one or more optical features, such as phase velocity, polarization, etc., changes in response to an acoustic wave. Fiber-based sensors may be based around optical fibers. A fiber-based sensor configured as an acoustic receiver may detect the acoustic wave by transmitting light through the optical paths and detecting changes in the transmitted light in response to changes in the one or more optical features. For example, a change in pressure on the fiber body induced by the acoustic wave can cause a change in refractive index and/or induce birefringence by deforming the fiber core. In another example, deformation or property change in response to a received acoustic wave of an optical resonator or interferometer disposed at a distal end of a fiber-based sensor may cause optical property changes.

As shown in FIGS. 2(a) and 2(b), various sensitive volumes 291 may be realized in a fiber-based sensor, which may provide different receiving directionality 293. Sensitive volumes 291 may include portions of the fiber sensor that are acoustically responsive. In some configurations, mirror coating and/or an acoustically responsive structure (e.g., a structure that deforms or has its properties altered in response to received acoustic waves) may be fabricated at the fiber tip, forming a resonant cavity to reflect an incident optical signal. Deformations in the resonant cavity or changes to the optical properties of the resonant cavity in response to incoming acoustic waves cause changes in the incident optical signal and therefore the reflected optical signal encodes acoustic sensing information. As shown in FIG. 2(a), a fiber sensor 274 may include such a sensor configuration and may provide near omnidirectional sensitivity except in a direction blocked or shaded by the fiber body.

In some configurations, an array of sensitive volumes may be distributed along a fiber. As shown in FIG. 2(b), a fiber sensor 275 may include such a sensor configuration and may provide a receiving directionality in the plane perpendicular to the fiber axis. It may be further noted that the representation of receiving directionality in both FIGS. 2(a) and 2(b) only shows the angular coverage of the sensitivity. In some configuration, no electrical links are required for a fiber-based sensor, because the optical fiber carries, and the array operation is realized by a tunable laser (array) in the back-end system.

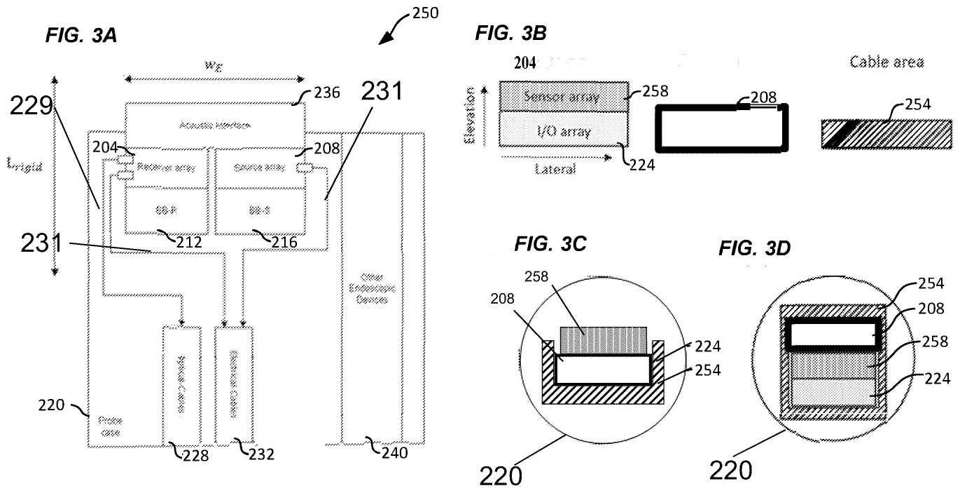

Referring now to FIGS. 3A-3D, sample designs of compact mixed ultrasound transducers of different types are described. A receiver array 204 (e.g., an ORA, as discussed above) and source array 208 (e.g., acoustic transducer array) may be bonded with backing blocks (BBs) 212 and 216 respectively and may be fixed inside a compact mixed ultrasound transducer housing or case 220 on a thermo-mechanical substrate. The thermo-mechanical substrate may be tailored to for disposition within the housing or case 220, which may be designed according to specific medical device applications. For example, an array on an end of an endoscopic catheter, the case 220 may be cylindrical in shape. An I/O array 224 may be included within the transducer case 220 as part of the receiver array 204. The I/O array 224 may provide a transition from the multiple optical sensors of the sensor array 258 of the receiver array 204 to the one or more optical links 228 and one or more electrical links 232. In embodiments, the receiver array 204 may require only optical links 228. In embodiments, the receiver array 204 may include one or more electrically powered or controlled devices, such as tuning devices, such as micro-heaters configured for tuning of the sensor elements in the optical receiver array 204. Such electrically powered or controlled devices may require the electrical links 232. The one or more optical links 228 and the one or more electrical links 232 are configured to carry electrical and/or optical signals from the array elements of the compact mixed ultrasound transducer to the proximal end of a medical device in which the compact mixed ultrasound transducer is disposed.

An acoustic interface 236 may be used to bridge an acoustic impedance mismatch between the source and receiver subarrays and an imaging target outside the probe. As used herein, “matching the impedance” may refer to selecting materials and/or structures that have acoustic impedances that match, generally it is known to those of skill in medical ultrasound that acoustic impedances within 20% of one another provide an acceptable match. The acoustic interface may be constructed of a medical-grade polymer such as RTV, Epoxy, ABS, particle-doped epoxy, etc. In embodiments, the acoustic interface may be configured with an acoustic impedance to match a local environment of use of the transducer 250 and may have a low acoustic attenuation.

Closer matches in acoustic impedance lead to a better transmission of the acoustic signal (e.g., a smaller portion of the acoustic signal is reflected) and thus higher sensitivity. The acoustic interface 236 may provide various functions, including but not limited to: (1) mechanical protection of subarrays; (2) management of the acoustic field of the source and the echo for better imaging reconstruction; and/or (3) creation of the acoustic channel between the subarrays to ensure acoustic conduction between the arrays and the region of interest in the imaging field. Though the acoustic interface 236 is shown as one component, it should be noted that the acoustic interface of the source array 208 and the receiver array 204 may not be the same component. Further, they may or may not be linked to each other. AEG transducers and optical sensors may require different impedance matching and thus the acoustic interface 236 may provide different impedance matching for each of the source array 208 and the receiver array 204. In embodiments, different impedances may be provided by different components of different materials, thicknessed, or shapes. In further embodiments, different impedances may be provided by a single integral component having different portions of differing materials, thicknesses, and/or shapes. In embodiments, the acoustic interface 236 may be shaped differently in portions covering the source array 208 vs the receiver array 204. For example, over the receiver array 204, the acoustic interface 236 may be a flat acoustic window while, over the source array, it may be shaped and configured as an acoustic lens.

One or more additional channels 240 (or lumens) may be provided to give passage to other endoscopic devices, such as one or more of an optical camera, an illumination source, and/or an aspiration device. In embodiments, the acoustic interface may be between 300 um-50 μm to accommodate ultrasound having a center frequency range from 3 MHz-20 MHz.

FIGS. 3A-3D illustrates a front-view design of a mixed ultrasound transducer in an example. FIG. 3A is a block diagram of an embodiment of a mixed ultrasound transducer 250. The mixed ultrasound transducer 250 includes optical links 228 and/or electrical links 232 from receiver array 204 and electrical links 232 from the source array 208. In embodiments, the optical links 228 may include optical waveguides, e.g. optical fibers, while the electrical links 232 In some configurations, the optical links 228 and/or electrical links 232 may be required to bend back sharply in the transducer case 220. Due to the compact geometry of a medical device within which the transducer case 220 is disposed, the transducer case 220 is also required to have a compact geometry. Disposition of the necessary optical links 228 and electrical links 232 within such a compact transducer case 220 may require the sharp bending described above. Sharp bending, as used herein, may include bending of 60° or more, 70° or more, 80° or more, or up to approximately 90°. Bending in optical waveguides may cause a reduction in signal to noise ratio due to an increase in insertion loss. Accordingly, to maintain performance, optical links 228 as described herein may be configured for sharp bending with reduced insertion loss. For example, in embodiments, heated bending of fibers may be employed to reduce insertion loss. In other examples, fibers with reduced core size may be used to reduce insertion loss related to bending. In further embodiments, a reflecting mirror may be introduced at a bend in the optical path, the reflecting mirror may be mode matched to ensure limited insertion loss.

FIG. 3A also illustrates the length L_{rigid} of the stacked acoustic interface 236, receiver array 204, sensor array 208, and backing blocks 212/216.

Acoustic (e.g., ultrasound) pulses are transmitted out of a front of the compact mixed ultrasound transducer 250, and reflected acoustic signals are received at a front of the compact mixed ultrasound transducer 250 (e.g., similar to the compact mixed ultrasound transducer shown in FIG. 1(a)). Further examples of mixed array designs are described in U.S. application Ser. No. 18/597,493, filed Mar. 6, 2024 titled MIXED ARRAY IMAGING PROBE and U.S. application Ser. No. 17/990,596, filed Nov. 18, 2022 for MIXED ULTRASOUND TRANSDUCER ARRAYS, the disclosures of which are incorporated by reference for all purposes.

FIGS. 3B-3D illustrate embodiments of cross-sectional designs for portions of the compact mixed ultrasound transducer 250, which show receiver array 204, source array 208, and cable area 254. The receiver array 204 comprises a sensor array 258 and an I/O array 224. The sensor array 258 may cover substantially the whole lateral width of the substrate or chip on which it is arranged, while the optical traces 229 and electrical traces 231 are routed along the side of the substrate or chip of the receiver array 204 (e.g., the ORA) as shown in FIG. 3A. While the optical traces 229 and electrical traces 231 are not shown in FIG. 3B, the layout capability enabled by lithography may limit the width of these traces to within less than a 0.2-millimeter extension or margin on either side of the ORA 204 and the source array 208. The optical traces 229 and electrical traces 231 may be deposited via lithography along the side of the substrate or chip to which the receiver array 204 and source array 208 are mounted and may extend out 0.2 mm or less, thereby enlarging the footprint by only a small amount. In some embodiments, these traces may overlap the with the sensor area and thereby cause no expansion to the footprint. The optical traces 229 and electrical traces 231 may connect to the optical links 228 and electrical links 232 within the transducer housing 220 for connection through to a proximal end of the medical device.

The cable area 254 represents a maximum additional area occupied by the optical traces 229 and electrical traces 231 and the optical links 228 and electrical links 232. As illustrated in FIGS. 3C and 3D, it can be seen that this area is kept to a minimum, thereby permitting a compact transducer size overall. In embodiments, the cross section of the cable area 254 may have a cross-sectional dimension (e.g., width, height, length, etc.) less than 10%, less than 15%, less than 20%, and/or less than 25% larger than a combined cross-sectional dimension of the receiver array 204 and source array 208.

FIGS. 3C and 3D show two different examples of a cross-section of the probe 250. As shown in FIG. 3C, the source array 208 may be bonded on the face of I/O array 224. This arrangement, which may be understood as a collocation of the receiver array 204 and the source array 208, reduces the footprint of the mixed ultrasound transducer. In this embodiment, the collocation of the receiver array and the source array includes overlapping of the elevational footprints of the receiver array 204 and the source array 208 without obscuring the sensors of each array. This arrangement may also introduce an axial offset between the source array 208 and the optical receiver array 204. The offset may be bridged, for example, by an acoustic window configured with low acoustic attenuation, reduced facet reflection, and/or acoustic focusing capabilities. Such an acoustic window may regulate the acoustic field front received by ORA 204 and may also flatten the geometry of the acoustic interface 236. The trace area of the receiver chip is shown as I/O array 224 in FIGS. 3B-3D. The embodiment shown in FIG. 3C is compact and may enable a larger elevation size of single elements in both subarrays. However, extra acoustic windows may be required in the acoustic interface 236 of the receiver array 204 to bridge a height difference. In FIG. 3D, the receiver array 204 and the source array 208 do not overlap in the cross-section.

The electrical cable (which may include flexible printed circuits) of the source array 208 may be configured so as to not cover the sensor array 258. The actual design of functional components of the I/O array 224 may occupy only a portion of the area labeled as I/O array 224 in FIGS. 3C-3D. In addition, the circular boundary shown in Figures FIGS. 3C-3D may represent only a minimum actual boundary of the transducer case 220 because the arrangement of the acoustic interface 236 and the channels 240 for other endoscopic devices may be specific to a particular application or medical device. That is, the arrangement of the acoustic interface 236, channels 240, and other aspects of the compact mixed ultrasound transducer may be selected to accommodate a medical device configured for a specific application, be it endoscopic, endoluminal, laparoscopic, etc. There may be more freedom in the design of medical devices including the mixed ultrasound transducer 250 because the compact nature of the transducer 250 permits more cross-sectional area to be devoted to other features, tools, etc. In embodiments, the transducer 250 may have a diameter in the range of approximately 1 to 8 mm, 1 to 6 mm, and/or 2 to 5 mm.

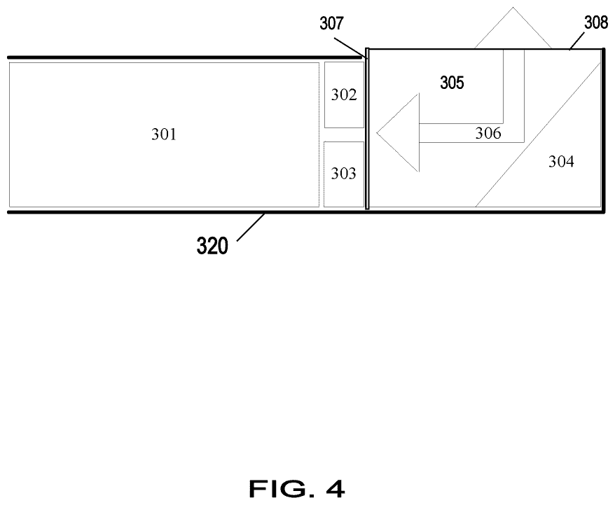

FIG. 4 illustrates an example of a compact mixed ultrasound transducer 350 including an acoustic interface and a source array. The components of FIG. 4 may be similar in nature and function to similar components described above, except where otherwise stated. Any and all features of the compact mixed ultrasound transducer 350 of FIG. 4 may also be utilized or combined with features of the compact mixed ultrasound transducer 250 shown in FIGS. 3A-3D. In the example shown, the acoustic energy source, such as a source array 302, outputs sufficient acoustic waves to insonify a region of imaging interest such that the optical receivers 303 may receive reflected acoustic wavefronts, e.g., echoes, for sensing purposes. As described above, common acoustic energy generators may be used (PZT, CMUT, PMUT, etc.) to form the source array 302. In embodiments, acoustic energy generators may be either single element or multi element arrays. The close proximity of the source array 302 and the optical receiver array 303 may be useful for performing improved optimal image construction. An example of an ultrasonic catheter is described in U.S. Pat. No. 5,876,345, issued on Mar. 2, 1999, which is incorporated by reference to provide an example of an ultrasonic transducer array.

The example shown in FIG. 4 includes a transducer case 320 and, disposed within the transducer case 320, a backing block 301, a source array 302 (e.g., similar to source array 208 in FIG. 2), and an optical receiver array 303 (e.g., similar to receiver array 204 in FIGS. 3(a)/3(b)). The source array 302 is mounted next to the optical array 303. In other examples, the optical receiver array 303 may be arranged differently in relation to the source array 302 such that the two may be integrated (e.g., to achieve close proximity between the two and also to reduce the overall size of the generator/receiver assembly in an endoscope). For example, as discussed above, the optical receiver array 303 and the source array 302 may be collocated such that elements of the optical receiver array 303 are interspersed with elements of the source array 302.

The example shown in FIG. 4 further includes an acoustic mirror 304 arranged in a coupling medium 305. The acoustic mirror 304 is configured to redirect the acoustic waves in the direction shown by arrow 306. Such a configuration may help with preventing the field of view (FOV) outside the compact mixed ultrasound transducer from suffering from an irregular acoustic field in the near field due to separating apertures because the near-field of the acoustic region lies within the coupling medium 305 inside the compact mixed ultrasound transducer 350. The coupling medium is a material that fills the illustrated space in the transducer. By adjusting the distance from the acoustic mirror 304 to the surface of the source array 302, the main near field may be passed within the compact mixed ultrasound transducer 350. This solution may be helpful in application scenarios where space is limited and/or the endoscopic target is expected to be located at the near field region. In some situations, the acoustic path within the coupling medium 305 may also introduce additional acoustic attenuation of both the transmitted and received (reflected) acoustic signal. The two extra boundaries at 307 and 308 may introduce reverberation. To suppress unwanted factors caused by these boundaries, a low-loss material with well-matched (e.g., within 20%) acoustic impedance at at least one of the boundaries may be included. For example, acoustic coupling may be realized by a liquid balloon at boundary 307 or boundary 308, having low acoustic attenuation. In some embodiments, the space occupied by the coupling medium 305 is open to the local environment of the compact mixed ultrasound transducer 350. This permits the coupling medium 305 to be or to be mixed with materials from the local environment. For example, the coupling medium 305 may be or may include blood. The coupling medium 305 may also include saline or water and, in some cases, air.

The configuration shown in FIG. 4 may allow the source array 302 and/or the optical receiver array 303 to be used in other compact mixed ultrasound transducer designs, such as a side or radial view application. For example, the structure shown in FIG. 4 allows a side view, but a similar structure without the mirror 304 may be used for a forward view application: the incoming acoustic signals would propagate axially through the coupling medium 305 towards the optical array 303. In examples where the configuration shown in FIG. 4 causes the signal intensity of a particular compact mixed ultrasound transducer to drop, e.g., due to acoustic attenuation within the coupling medium 305, the drop can be compensated for by a corresponding increase in the sensitivity of the optical receiver array 303.

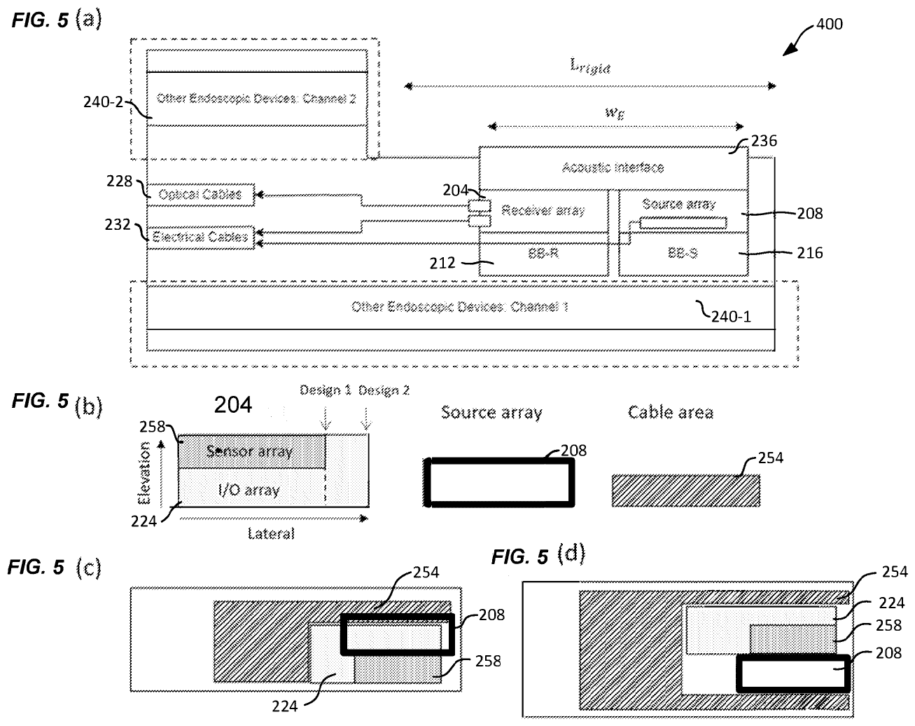

FIGS. 5(a)-5(d) illustrate embodiments of a compact mixed ultrasound transducer 400 configured for side view imaging. The components of FIGS. 5(a)-5(d) may be similar in nature and function to similar components described above, except where otherwise stated. Any and all features of the compact mixed ultrasound transducer 400 may be combined and/or otherwise included with other compact mixed ultrasound transducer designs disclosed herein. The compact mixed ultrasound transducer 400 may include a first channel 240-1 and a second channel 240-2 to accommodate one or more other endoscopic devices. Channels 240-1/240-2 are optional. For example, the second channel 240-2 may be utilized in an aspiration biopsy to insert a biopsy needle. In this way, once the biopsy needle is inserted through the second channel 240-2, it would be visible in the ultrasound imaging. The compact mixed ultrasound transducer 400 may include an acoustic interface 236, a receiver array 204, a source array 208, backing blocks 212 and 216, optical links 228, and electrical links 232. The compact mixed ultrasound transducer 400 is configured such that the optical and/or electrical routing does not have sharp bends as in the compact mixed ultrasound transducer 250.

FIGS. 5(b)-(d) depict embodiments of a transducer component layout, which may include the receiver array 204 having a sensor array 258 and an I/O array 224, the source array 208, and a cable area 254. The component layout of the receiver array 204 as illustrated in FIG. 5(b) differs from that of FIG. 3(b). The differences may stem from the design of the I/O array 224. As shown in FIG. 5(b), the I/O array 224 extends laterally further than the sensor array 258. FIGS. 5(b)-(d) illustrate embodiments of the cross-sectional designs of the probe schematic of the optical receiver array 204 shown in FIG. 5(b). The sensor array 258 may cover substantially the whole lateral width of the I/O array 224 (Design 1), or only a portion (Design 2). Optical and electrical traces may be routed along and around the side of the receiver array 204 as shown in FIG. 3(a) or FIG. 5(a), may be routed along one side as shown in FIG. 5(c), or both sides as in FIG. 5(d). FIGS. 5(c) and 5(d) show two different floorplan examples of the cross-section of the compact mixed ultrasound transducer 400. In FIG. 5(c), the source array 208 is bonded atop the I/O array 224 of the optical receiver array 204, meaning that the source array 208 and the receiver array 204 may be collocated and may overlap in cross-section. The source array 208 may be bonded atop the receiver array 204 such that the source array 208 does not overlap the sensor array 258. In this embodiment, the collocation of the receiver array and the source array includes overlapping of the elevational footprints of the receiver array 204 and the source array 208 without obscuring the sensors of each array. In another embodiment, as in FIG. 5(d), the source array 208 and the receiver array 204 do not overlap in the cross-section. In embodiments, the transducer 400 may have a diameter in the range of approximately 2 to 12 mm, 2 to 6 mm, 2 to 5 mm, and/or 2-4 mm. The transducer 400 may, for example, have a diameter smaller than that of transducer 250, due to the layout of the receiver array 204 and the source array 208 wherein the lateral dimension extends axially with respect to the transducer case.

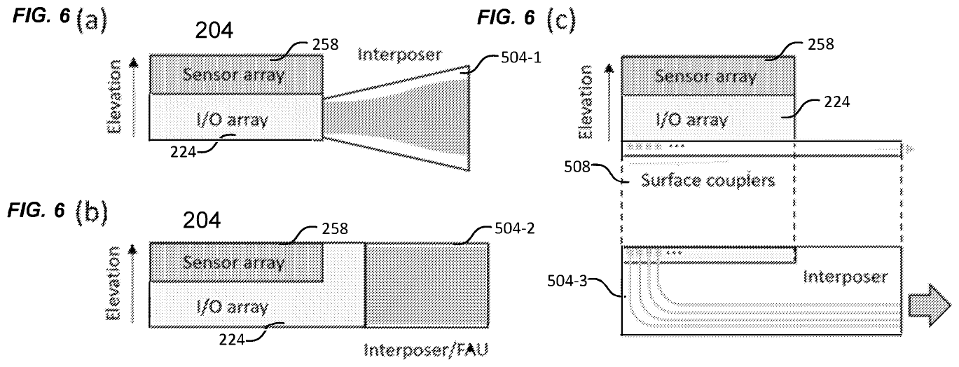

FIGS. 6A-C illustrates three example configurations of the optical I/O array 224 corresponding to the side-view compact mixed ultrasound transducer designs described in relation to FIGS. 5A-D. FIGS. 6A-6C illustrate a sensor array 258 and an I/O array 224 in relation to an interposer 504. The examples shown in FIGS. 6(a) and 6(b) utilize edge coupling between the I/O array 224 and the interposer 504-1/504-2. In further examples, a surface coupler 508 may be used to form a two-dimensional (2D) coupler array, such as illustrated in FIG. 6(c). Given a fixed cable orientation in the transducer case, edge couplers and surface couplers may provide different design options. In some embodiments, for example, as in FIGS. 6(a) and 6(b) interposers may be provided to eliminate sharp bending in the optical cables within the transducer case by providing the necessary routing within the interposer.

FIGS. 6(a) and 6(b) illustrate examples with edge couplers on the elevation side of the optical receiver array 204. In the example shown in FIG. 5(a), the sensor array 258 extends the lateral width of the I/O array 224, leaving only partial elevation sides for edge coupling with the I/O array 224 using a first interposer 504-1. The first interposer 504-1 (e.g., an interposer chip) may be utilized with the 1/0 array 224 without adding significant additional elevation and may function to couple the relevant input and output channels from the I/O array 224 to inputs of a fiber array unit to carry the signals to the proximal end of the medical device incorporating the mixed ultrasound transducer array. The first interposer 504-1 is configured to broaden or spread the input and output channels of the I/O array 224. In some examples, the interposer may be based on CMOS-compatible PIC platform or planar lightwave circuits (PLCs) based on glass substrates. The first interposer 504-1 may provide advantages due to its compact footprint.

In the example shown in FIG. 6(b), the I/O array 224 is elongated laterally at an edge of the sensor array 258 to allow I/O array 224 to occupy the whole elevation on one side, which may enable coupling with a second interposer 504-2 or may enable an FAU to directly attach to the I/O array 224 without use of the interposer 504-2. The second interposer 504-2 has a larger elevation height because the I/O array 224 array 224 in FIG. 6(b) has a larger elevation height. The second interposer 504-2, although it has a larger footprint than the interposer 504-1, may provide an advantage in that the larger coupling edge may permit a larger tolerance in the pitch of the input/output connections.

In some examples where the elevation size of the probe is limited, edge coupling on the elevation side may be challenging due to the limited space within which to make the connections. In such examples, the interposer 504 may be moved to the lateral side. In this way the elevation size of the I/O array 224 might be limited to, for example, under 0.5 mm. However, such an example may also require sharp bending of optical links, as described above and as depicted in FIG. 6(c). Such a configuration may use a relatively complex interposer chip shape. FIG. 6(c) provides an example of coupling to the I/O array 224 on the lateral side using surface couplers. As shown in FIG. 6(c), the third interposer 504-3 may include internal routing to provide a sharp (e.g., 60° or more, 70° or more, 80° or more, or 90°) bend to accommodate the surface coupling arrangement. In FIG. 6(c), a third interposer 504-3 and surface couplers 508 are shown. Rather than arrange the third interposer 504-3 in the same plane as the receiver array 204 (e.g., as shown in FIGS. 6(a) and 6(b)), the third interposer 504-3 is mated with the I/O array 224 in the elevation dimension (e.g. vertically) via right-angle surface couplers 508, in the example shown in FIG. 6(c). In some configurations, the third interposer 504-3 is fabricated using glass-based PLC, and the right-angle surface coupler 508 comprises a side-coupled spot-size converter with a 45-degree-angle mirror.

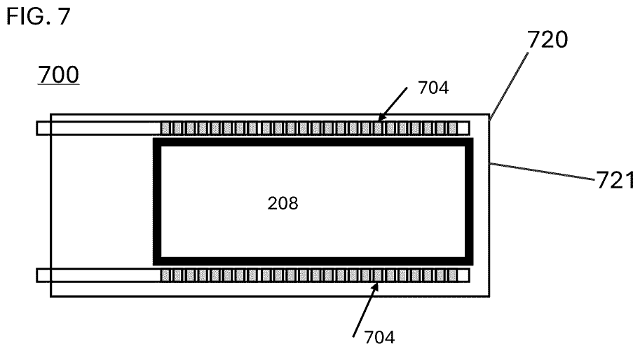

FIG. 7 illustrates a side view compact mixed ultrasound transducer including a fiber-based sensor array. The components of FIG. 7 may be similar in nature and function to similar components described above, except where otherwise stated. The compact mixed ultrasound transducer 700 includes features that may be combined with any or all of the features of other compact mixed ultrasound transducer embodiments discussed herein. The compact mixed ultrasound transducer 700 includes a transducer case 720, a source array 208, and a one or more fiber-based ORAs 704. The compact mixed ultrasound transducer 700 includes a distal face 721 disposed perpendicularly to an axis of extension of the compact mixed ultrasound transducer 700. As illustrated in FIG. 7, the compact mixed ultrasound transducer 700 may be disposed in a medical device (e.g., catheter) that extends laterally to the left as shown in the image. One, two, or even more of 1D ORA 704 may be incorporated with an elevational offset to the source array 208 (e.g., the AEG array). The fiber sensor used in this embodiment as the ORA 704 may be similar to the sensor 275 as shown in FIG. 2(b). In this embodiment, the ORA 704 has multiple acoustically sensitive volumes extending along its length. Due to the sequential nature of these sensitive volumes, the cable management may be simplified and may only require a number of fibers that corresponds to the number of ORAs 704 employed, rather than a number of fibers that corresponds to the number of sensors. As discussed above, an AEG transducer array may be approximately 1.1-1.2 mm in the elevation dimension and a single optical fiber transducer may be between 50-250 um in diameter. Accordingly, the size (diameter) of the compact mixed ultrasound transducer 700, when the transducer case 720 is included, may be between approximately 1.4 mm and 2.5 mm, or between 1.2 mm and 2 mm.

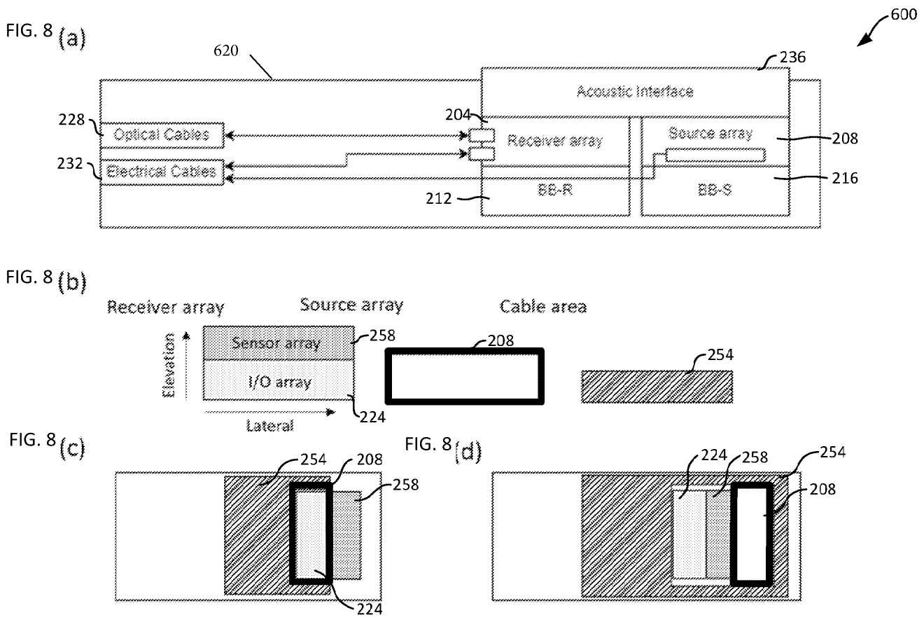

FIGS. 8(a)-8(d) illustrate an example designs of a radial-view compact mixed ultrasound transducer 600. The components of FIGS. 8(a)-8(d) may be similar in nature and function to similar components described above, except where otherwise stated. The compact mixed ultrasound transducer 600 may be similar to the compact mixed ultrasound transducer 400 of FIG. 5(a) in that an acoustic interface 236 is disposed on a lateral side rather than a distal end face of the transducer case 620. As illustrated, the compact mixed ultrasound transducer 600 does not include endoscopic channels, although some configurations may include one or more endoscopic channels. Some examples may include an array of imaging elements (e.g., a source array 208 and a receiver array 204). However, in some examples, only one imaging element (e.g., a single imaging sensor may comprise the receiver array 204) may be used because rotational scanning can realize imaging beamforming with a single-element probe. The compact mixed ultrasound transducer 600 may include an acoustic interface 236, a receiver array 204, a source array 208, backing blocks 212 and 216, optical links 228, and electrical links 232. Due to the side facing arrangement of the receiver array 204 and the source array 208, it may not be necessary to include sharply bending links in the I/O design for the radial-view transducer. In some applications, the compact mixed ultrasound transducer 600 may have a very small footprint. For instance, in applications like intravascular ultrasound (IVUS), a transducer case 620 housing the compact mixed ultrasound transducer 600 may have a diameter of approximately 1 mm. Accordingly, some examples of such reduced footprint transducers may include optical receiver arrays 204 (having a single or multiple elements) that do not require electrical control and therefore may provide for a channel count in the I/O array 224 as small as 8 or even fewer. In such an embodiment, each optical sensor element may be driven by a specific independent laser. In embodiments, wavelength division multiplexing may be employed in optical cables 228 that extend the length of the medical device incorporating the compact mixed ultrasound transducer 600, and therefore the number of optical cables 228 may be reduced to better fit in the transducer case 620. Wavelength division multiplexing serves to permit a single optical fiber to carry multiple input and output signals with minimal interference. Wavelength division multiplexing may be applied to any of the transducer embodiments disclosed herein.

FIGS. 8(b)-8(d) illustrate the cross-sectional design of the compact mixed ultrasound transducer 600. FIGS. 8(b)-8(d) illustrate a receiver array 204 having a sensor array 258, and an I/O array 224, a source array 208, and a cable area 254. The sensor array 258 may extend a lateral width of the I/O array 224. Optical and electrical traces are routed along a side (e.g., similar to those shown and described in relation to FIGS. 3(c) and 3(d)), e.g., through the cable area 254. FIG. 8(c) illustrates an embodiment of a compact mixed ultrasound transducer 600 wherein the source array 208 is bonded over the I/O array 224, thereby serving to reduce the overall footprint of the array layout. In this embodiment, the collocation of the receiver array and the source array includes overlapping of the elevational footprints of the receiver array 204 and the source array 208 without obscuring the sensors of each array. FIG. 8(d) illustrates an embodiment wherein the source array 208 does not overlap the I/O array 224.

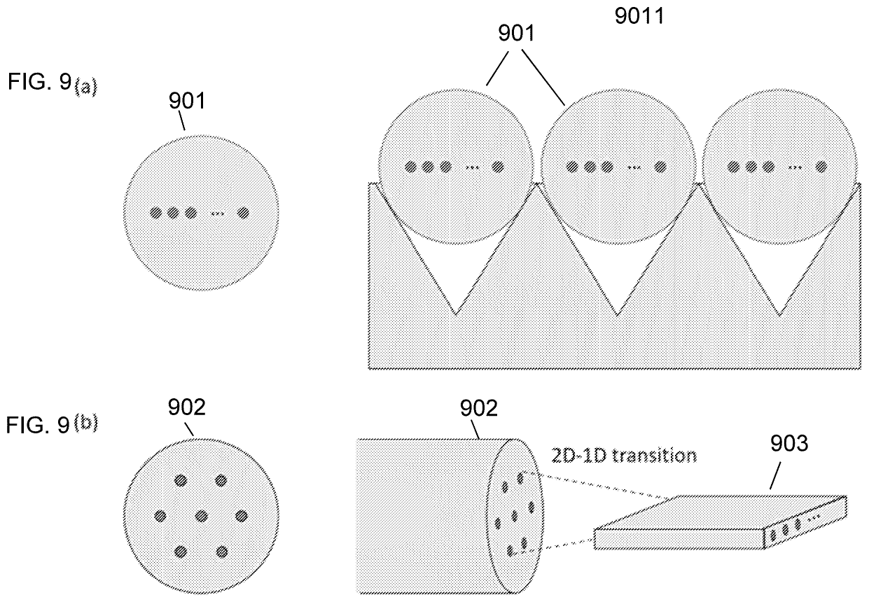

Though some configurations may use single-core fibers for the optical input and output (e.g. the optical cables 228), other configurations may use a multi-core fiber (MCF) for the optical input and output (e.g., to reduce an optical I/O footprint and/or a total diameter of an optical-fiber bundle for endoscopic applications). FIGS. 9(a) and 9(b) illustrates examples of multi-core fibers. The MCFs of FIGS. 9(a) and 9(b) may be combined with any of the embodiments discussed herein.

FIG. 9(a) shows a linear MCF 901 and a fiber array 9011 formed from a plurality of linear MCFs 901. The linear MCF 901 may support (e.g., act as the optical cabling 228 for) an optical receiver array 204 (e.g., with a small channel count) with one single fiber. A multicore fiber array unit (FAU) of an optical receiver 204 may be mated to a linear MCF 901 via an interposer configured to narrow the channel pitch. Such an FAU configuration may in some cases lead to a non-periodical coupler array and a particular rotational alignment during the FAU manufacturing. As a result, use of one or more n-core MCFs may reduce the channel count of the optical cabling 228 by n times. Because optical fibers may be bulky with cladding layers and protection coating, the introduction of the linear MCF 901 may simplify cable management for embodiments requiring either a large optical receiver count for the optical receiver array 204 (especially front-view and side-view) or a small catheter diameter. This arrangement may be particularly advantageous in a front-view and or radial-view transducer configuration.

FIG. 9(b) illustrates an MCF 902 that is rotationally symmetric in the cross-section in one example. The rotationally symmetric MCF 902 permits miniaturization of the optical cables 228 and is compatible with any of the embodiments disclosed herein. In some examples in which the optical receiver array 204 includes a 1-D array of couplers 903, a 2D-1D transition may be used within the transducer case. For example, optical wire-bonding or a 3D waveguide direct writing may be utilized for the transition.

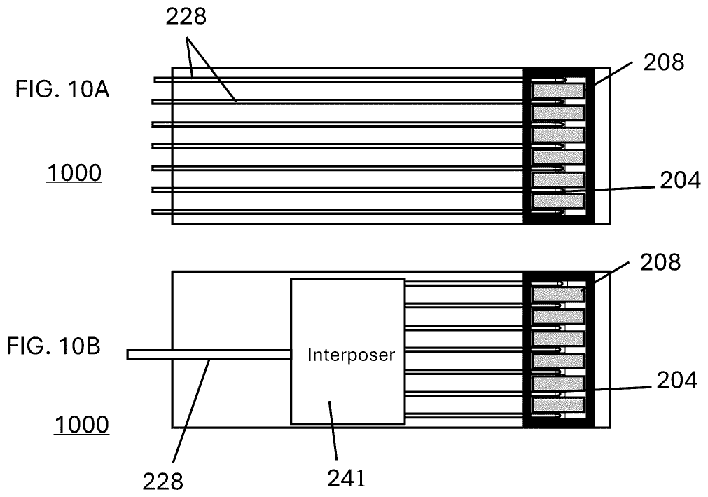

FIGS. 10A and 10B illustrate an example of a side-view compact mixed ultrasound transducer 1000 including a fiber-based ORA 204 collocated with a source array 208. Features illustrated in FIGS. 10A and 10B may be combined with any compact mixed ultrasound transducer embodiments disclosed herein. Individual fiber sensors of the fiber-based ORA 204 may be disposed within the kerf of the source array 208. The individual fiber sensors may be arranged between the individual AEG elements of the source array 208. Thus, the ORA 204 and the source array 208 may be collocated to reduce a footprint of the compact mixed ultrasound transducer 1000. In this example of collocation, the individual fiber sensors of the fiber-based ORA 204 are interspersed with the AEG elements of the source array 208. In an embodiment, as shown in FIG. 10A optical cables 228 may include a same number of optical fibers as sensors in the ORA 204. In a further embodiment, as shown in FIG. 10B, an interposer 241 may be employed. The interposer 241 may be configured to reduce the number of channels from the ORA 204 such that fewer optical channels may be used in the optical cabling (e.g., through the use of WDM) or may be configured to transition the multiple channels of the ORA 204 into a multicore optical fiber.

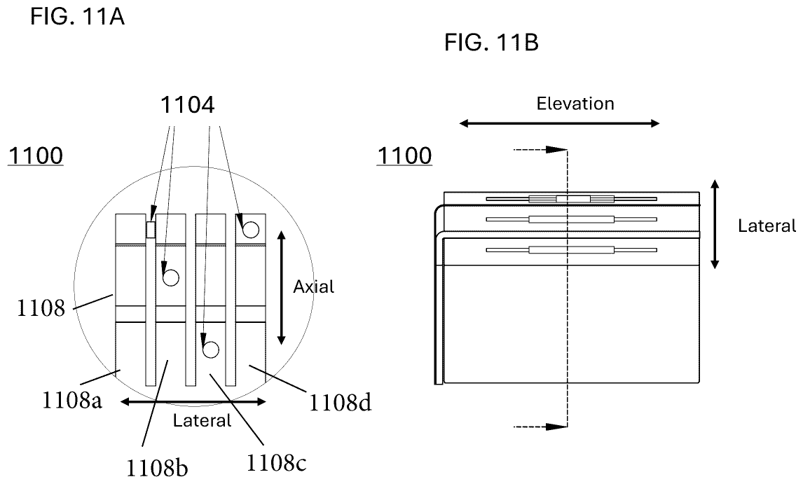

FIGS. 11A and 11B illustrate an example of a front-view compact mixed ultrasound transducer 1100 including a fiber based ORA 1104 collocated with a source array 1108. Features of the compact mixed ultrasound transducer 1100 may be combined with any of the other embodiments discussed herein. FIGS. 11A and 11B illustrate a representative portion of a compact mixed ultrasound transducer 1100. The position and number of the individual AEG elements 1108a, 1108b, 1108c, 1108d and the position and number of the individual optical fiber sensors of the optical receiver array 1104 are provided by way of example. The individual sensor fibers of the ORA 1104 may be arranged in a collocated and interspersed arrangement with respect to the individual AEG transducers of the source array 1108 FIG. 11A illustrates a cross sectional view of the compact mixed ultrasound transducer 1100. The source array 1108 includes individual AEG elements 1108a, 1108b, 1108c, 1108d. As illustrated in the FIG. 11A, the individual fibers of the optical receiver array 1104 may be interspersed with the elements of the source array 1108 in any suitable location, including in between AEG elements, embedded within the active acoustic generator material of AEG elements, embedded within the backing blocks of the AEG elements, and/or within a matching layer of the source array 1108. FIG. 11B illustrates a side view of the compact mixed ultrasound transducer 1100. Collocation of the generator and receiver may allow an optimum transmit-receive response to be attained and may further function to reduce the overall footprint of the compact mixed ultrasound transducer 1100.

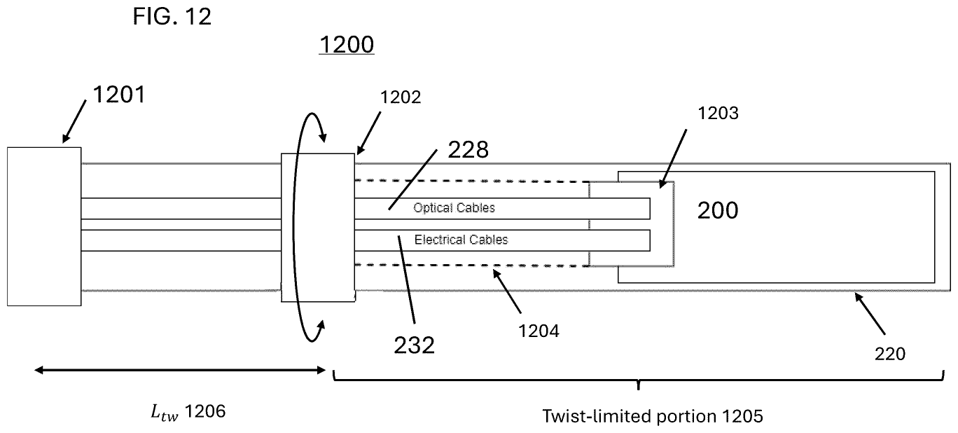

FIG. 12 illustrates a rotational strain relief device 1200 compatible with embodiments hereof. In some applications, such as intravascular ultrasound catheters, inserted medical devices are configured for rotation. Rotational torque/strain on optical cables compatible with embodiments hereof may cause artifacts or other errors in optical signals transmitted by the optical cables. The rotational strain relief device 1200 is configured to reduce rotational strain or torque on the optical cables 228. The rotational strain relief device 1200 includes a system connection device 1201, a proximal cable strain relief device 1202, a distal cable strain relief device 1203, and a rotation transmission fixture 1204. The rotational strain relief device 1200 is shown providing strain relief to a medical device that includes the compact mixed ultrasound transducer 200 within a transducer case 220 and having optical cables 228 and electrical cables 232. The distal cable strain relief 1203 provides mechanical support to the attachment between cables 228/232 and the transducer 200. The cables 228/232 are fixed with the proximal cable strain relief device 1202, which protrudes outside of the catheter case. During rotation, the drive (e.g., torque)) is applied to the outer rim of the proximal strain relief device 1202. The rotation transmission fixture 1204 links the proximal cable strain relief device 1202 to the distal cable strain relief 1203 and the transducer 200 to permit rotation of the mixed ultrasound transducer 200 without applying significant torque or rotation to the either cable-transducer attachment point or to the cables 228/232. For example, the rotation transmission fixture 1204 may be an inner catheter where the transducer 200 is mounted and fixed to the distal strain relief 1203. The twist-limited portion 1205 of the medical device is defined as the portion distal of the proximal cable strain relief device 1203, where the optical cables 228 and the electrical cables 232 are not exposed to significant twisting. Instead, the twisting (e.g., rotational strain) is spread across the length L_tw 1206 between the proximal cable strain relief device 1203 and the system connection device 1201. The system connection device 1201 to the back-end system may include a fixed cable strain relief to isolate the in-system cables from any outside interruptions. By spreading any rotation across the entire length L_tw 1206, the rotational strain may be distributed such that it does not create significant artifacts or noise within the optical cables 228 and the electrical cables 232. The maximum continuous rotation angle may be a function of the length L_tw 1206.

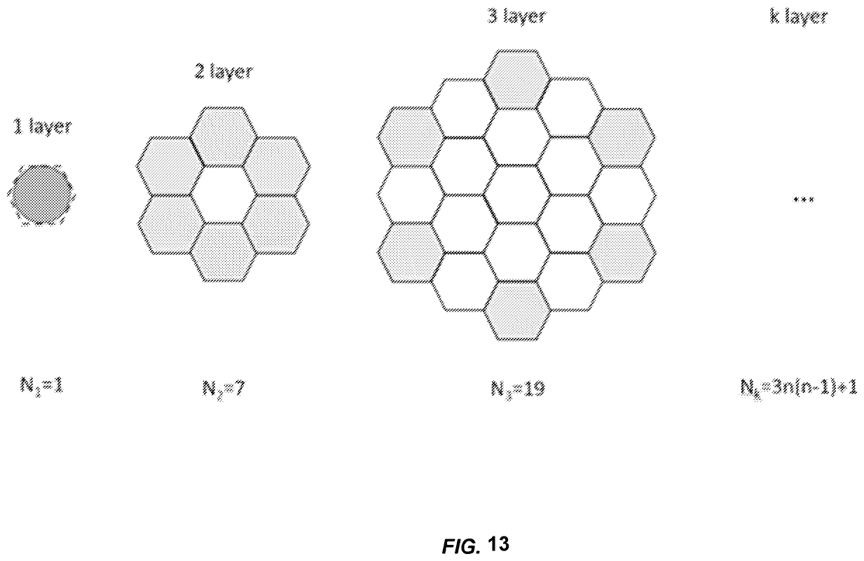

In embodiments, optical cables are bundled in the transducer case (e.g., transducer case 220, 620, etc. disposed at a distal end of a medical device). As discussed above, reducing the size of the optical cables 228 may be advantageous for various medical device uses. To estimate a bundle size of a plurality of individual cables, the fibers may be assumed to be densely distributed in a hexagonal way. FIG. 13 illustrates a circumscribed hexagon of a fiber bundle, which represents the area occupied by fiber bundles. In examples shown in FIG. 10, for bundles containing different layers, the farthest fiber is labeled in gray. A minimum diameter of the fiber bundle based on varying numbers of fibers is summarized in Table 1.

| | Fiber Count N | | | | |
|---|---|---|---|---|---|
| | 1 | 7 | 19 | 37 | 61 |
| | Bundle Diameter D | | | | |
| | d | 3 d | 5 d | 7 d | 9 d |
| d = 0.25 mm | 0.25 | 0.75 | 1.25 | 1.75 | 2.25 |
| d = 0.125 mm | 0.125 | 0.375 | 0.625 | 0.875 | 1.125 |
| d = 0.1 mm | 0.1 | 0.3 | 0.5 | 0.7 | 0.9 |
| d = .08 mm | 0.08 | 0.24 | 0.4 | 0.56 | 0.72 |

### Table 1. Estimation of Fiber Bundle Diameter

In the examples shown in FIG. 13 and Table 1, the fiber count N may not be directly related to the sensor channel count. For instance, in examples that utilize WDM and/or multicore fibers, a sensor channel count may be multiple times a size of N.

In some examples, to facilitate the constrained dimensions of the medical device delivery, fine wire (coaxial or twisted pair) or flexible circuit cabling may be utilized as the electrical cable to fit the confines of the delivering catheter.

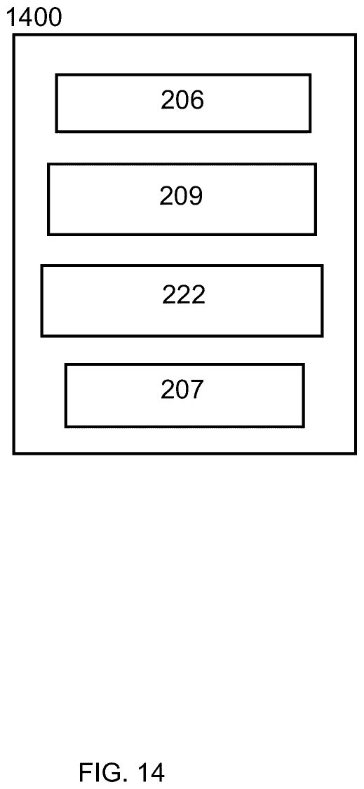

FIG. 14 illustrates an example ultrasound system compatible with embodiments hereof. The processing system 1400 may include a processing unit 209, an image reconstruction unit 206, an source array control unit 222 and a receiver array control unit 207. The processing system 1400 is provided by way of example only and it will be understood that similar functionality may be achieved through the use of different components. The processing unit 209 may include at least one computer processor, at least one non-transitory computer readable storage medium, and appropriate software instructions. The processing unit 209 is configured to provide control signals to and receive information signals from image reconstruction unit 206, an source array control unit 222 and a receiver array control unit 207. The processing unit 209 may communicate (via control signals and information signals) with the receiver array control unit 207, thereby providing control of optical signals provided to the fiber optical sensors of the optical receiver arrays described herein. The processing unit 209 may communicate (via control signals and information signals) with the source array control unit 222, thereby providing control and reception of acoustic signals via the source arrays described herein. Thus, processing unit 209 operates to provide the necessary control signals and receive the acquired information signals from the mixed ultrasound transducers described herein.

The processing unit 209 is further in communication with the image reconstruction unit 206, which operates to generate images based on the data and/or information acquired by the processing unit 209. The image reconstruction unit 206 may generate images based on data related to a medium, such as a human body, captured by the optical receiver arrays of the mixed ultrasound transducers described herein. The mixed ultrasound transducers may be incorporated into medical devices, including but not limited to, needles, a catheters (e.g., endoluminal catheters, endoscopic catheters, etc.), guidewires, delivery devices, and/or any other device or apparatus configured for use within the body of a patient. The image reconstruction unit 206 may be integrated within a system containing the processing unit 209 and/or may be a separate system including at least one computer processor, at least one non-transitory computer readable storage medium, and appropriate software instructions. The processing system 1400 may provide control signals to an output device to provide a data output. The output device may include, for example, a display or a device including a display. The output device may provide output, e.g., in the form of images produced by the image reconstruction unit 206 and/or in the form of any other data or information generated by the processing unit 209.

In some embodiments, the output device may further include additional systems, such as a medical procedure system that is configured to use the data that is output. For example, an output device may include an endoscopy system, a laparoscopic system, a robotic surgical system, neurosurgical system and additionally may include an interoperative ultrasound imaging system. The output data may include information about a location of the medical device distal end, and images acquired of the medium in the area of where the medical device distal end is used/deployed such as the patient anatomy, tissues, other medical tools/devices etc.

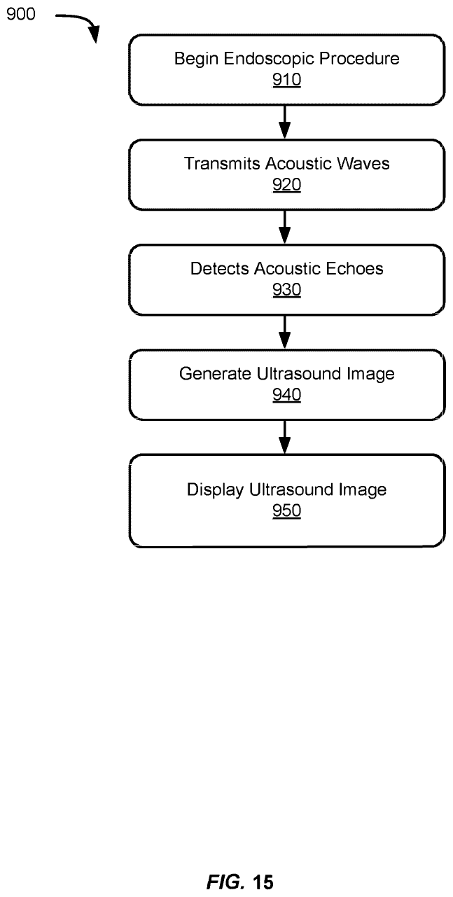

FIG. 15 illustrates a method 900 of using a mixed ultrasound transducer in some examples. At block 910 in the example shown, a medical professional begins an endoscopic procedure (or other medical procedure) by, for example, inserting an endoscope (or other medical device) into a patient. The endoscope includes a compact mixed ultrasound transducer as described herein.

At block 920, the compact mixed ultrasound transducer (e.g., the source array 208) generates and transmits acoustic waves. The acoustic waves are reflected back to the compact mixed ultrasound transducer by objects, such as tissue, in the path of the waves in the form of echoes.

At block 930, the compact mixed ultrasound transducer (e.g., the receiving array 204) detects the echoes. The compact mixed ultrasound transducer then transmits one or more signals representing the echoes to a processor. At block 940, the processor generates an ultrasound image. At block 950, the ultrasound image is displayed.

The foregoing description of example embodiments has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an embodiment, example, or implementation means that a particular feature, structure, operation, or other characteristic described in connection with the example may be included in at least one implementation of the disclosure. The disclosure is not restricted to the particular examples or implementations described as such. The appearance of the phrases “in an embodiment,” “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and Conly; Band C only; and A and B and C.

That which is claimed is:

1. A compact mixed ultrasound transducer comprising:
a transducer case;
a source array configured comprising at least one acoustic energy generating transducer and configured to transmit acoustic waves; and
an optical receiver array including at least one optical sensor and configured to detect acoustic echoes associated with the acoustic waves.

2. The compact mixed ultrasound transducer of claim 1, wherein the source array and the optical receiver array are collocated.

3. The compact mixed ultrasound transducer of claim 2, wherein collocation of the optical receiver array and the source array includes interspersal of the at least one acoustic energy generating transducer and the at least one optical sensor.

4. The compact mixed ultrasound transducer of claim 2, wherein collocation of the optical receiver array and the source array includes overlapping of elevational footprints.

5. The compact mixed ultrasound transducer of claim 1, wherein the optical receiver array includes 30 to 48 optical receiving elements and has a lateral dimension of approximately 3 mm to 4.8 mm.

6. The compact mixed ultrasound transducer of claim 1, wherein the source array includes 30 to 48 optical receiving elements and has a lateral dimension of approximately 3 mm to 4.8 mm.

7. The compact mixed ultrasound transducer of claim 1, wherein the transducer case has a diameter between 2 and 5 mm.

8. The compact mixed ultrasound transducer of claim 1, wherein the transducer case includes a rigid length of between 2 and 5 mm.

9. The compact mixed ultrasound transducer of claim 1, further comprising an acoustic interface coupled to at least one of the source array and the optical receiver array.

10. The compact mixed ultrasound transducer of claim 9, wherein the acoustic interface includes a first portion configured as an acoustic lens for the source array and a second portion configured as an acoustic window for the optical receiver array.

11. The compact mixed ultrasound transducer of claim 1, further comprising an acoustic mirror configured to redirect the acoustic waves.

12. The compact mixed ultrasound transducer of claim 11, wherein the transducer case is configured to accommodate a coupling medium between the source array and the acoustic mirror.

13. The compact mixed ultrasound transducer of claim 1, wherein the source array and the optical receiver array are a first mixed-array transducer, the compact mixed ultrasound transducer further comprising a second-mixed-array transducer coupled to an endoscope.

14. The compact mixed ultrasound transducer of claim 1, wherein the source array and the optical receiver array are configured to capture forward view ultrasound information.

15. The compact mixed ultrasound transducer of claim 1, wherein the source array and the optical receiver array are configured to capture side view ultrasound information.

16. The compact mixed ultrasound transducer of claim 1, wherein the source array and the optical receiver array are configured to capture radial view ultrasound information.

17. The compact mixed ultrasound transducer of claim 1, wherein the transducer case is configured for incorporation within an endoscope.

18. The compact mixed ultrasound transducer of claim 1, further comprising a multi-core optical fiber configured to provide input and output signals to the optical receiver array.

19. The compact mixed ultrasound transducer of claim 1, further comprising an optical fiber configured for wavelength division multiplexing to provide input and output signals to the optical receiver array.

20. The compact mixed ultrasound transducer of claim 1, further comprising in interposer configured to provide a transition between one or more optical cables and an input/output array of the optical receiver array.

21. The compact mixed ultrasound transducer of claim 20, wherein the interposer is edge coupled to the input/output array.

22. The compact mixed ultrasound transducer of claim 20, wherein the interposer is surface coupled to the input/output array.

23. The compact mixed ultrasound transducer of claim 1, further comprising a cable area configured to accommodate inputs and outputs to the optical receiver array and the source array, the cable area having a cross-sectional dimension less than 25% larger than a combined cross-sectional dimension of the optical receiver array and the source array.

24. The compact mixed ultrasound transducer of claim 1, further comprising:
a processor in communication with the source array and the optical receiver array and operable to generate an ultrasound image; and
a display in communication with the processor and operable to display the ultrasound image.
